(19) United States
(12) Patent Application Publication
(10) Pub. No.: US 2013/0217977 A9
(43) Pub. Date: Aug. 22, 2013

(54) SYSTEM, BUSINESS AND TECHNICAL METHODS, AND ARTICLE OF MANUFACTURE FOR DESIGN, IMPLEMENTATION, AND USAGE OF BIOMETRIC, PROXIMITY, AND OTHER SENSORS TO DETECT, RECORD, AND TREAT PERSONS THAT MAY BE OR HAVE BEEN INVOLVED IN CERTAIN PHYSICAL INJURIES OR DISABILITIES

(71) Applicant: Jason Ryan Cooner, Birmingham, AL (US)

(72) Inventor: Jason Ryan Cooner, Birmingham, AL (US)

(21) Appl. No.: 13/219,605

(22) Filed: Aug. 27, 2011

Related U.S. Application Data
(60) Provisional application No. 61/378,494, filed on Aug. 31, 2010.
(60) Provisional application No. 61/388,186, filed on Sep. 30, 2010.
(60) Provisional application No. 61/453,197, filed on Mar. 16, 2011.

(51) Int. Cl. A61B 5/0476; A61B 5/11

(52) U.S. Cl. 600/301; 600/595

(57) ABSTRACT

Non-invasive brain and body injury and vital sign assessment monitors, as well as methods for providing Internet-enabled care and recovery services for related conditions and injuries are disclosed. The sensors may be enclosed in a head wrap known as a “skull cap”, or they may be worn on other parts of the body such as the wrist or ankle. The Internet-enabled care systems related to injuries are intended to provide a step-by-step process for providing care and recovery services, as well as coordinating all stakeholders through the care and recovery process. Stakeholders include the athlete, parent or guardian for youth sport players, coach, educators or teachers, physician and/or athletic trainers.

### FIELD OF THE INVENTION

The present invention relates to brain and body assessment monitors, and relates to detection of brain trauma, stroke, and other related injuries sustained during physical activity. The invention also covers providing Internet enabled healthcare provider care associated with such injuries as a consolidated system. Other biometric sensor arrays including those designed to measure vitals and provide remote care for cardiovascular and neurocognitive disorders are included as part of the invention.

### BACKGROUND

Traumatic brain injury (TBI) is a serious public health problem in the United States. Each year, traumatic brain injuries contribute to a substantial number of deaths and cases of permanent disability. A TBI is caused by a bump, blow or jolt to the head or a penetrating head injury that disrupts the normal function of the brain. The severity of a TBI may range from “mild,” i.e., a brief change in mental status or consciousness to “severe,” i.e., an extended period of unconsciousness or amnesia after the injury. The majority of TBIs that occur each year are concussions or other forms of mild TBI. A concussion is a type of TBI that is caused by a bump, blow, or jolt to the head that can change the way your brain normally works. Concussions can also occur from a fall or a blow to the body that causes the head and brain to move quickly back and forth. Healthcare professionals may describe a concussion as a “mild” brain injury because concussions are usually not life-threatening. Even so, their effects can be serious. Concussions may be caused by a blow to the head or by acceleration forces without a direct impact. The forces involved disrupt cellular processes in the brain for days or weeks.

TBI can cause a wide range of functional short- or long-term changes affecting thinking, sensation, language, or emotions. Side effects can include:
Headache, a feeling of “pressure in the head”
Neck pain
Balance problems, dizziness
Nausea, vomiting
Vision problems
Hearing problems/ringing in ears
Feeling “dinged” or “dazed”
Confusion, feeling as though “in a fog”
Drowsiness, fatigue
More emotional than usual, irritability
Difficulty concentrating or remembering.
Later symptoms may include:
Sadness, nervousness or anxiety
Trouble falling asleep
Sleeping more than usual
Sensitivity to light or noise

TBI can also cause epilepsy and increase the risk for conditions such as Alzheimer's disease, Parkinson's disease, Lou Gehrig's disease and other brain disorders that become more prevalent with age. Repeated mild TBIs occurring over an extended period of time (i.e., months, years) can result in cumulative neurological and cognitive deficits. Repeated mild TBIs occurring within a short period of time (i.e., hours, days, or weeks) can be catastrophic or fatal.

The signs and symptoms of a concussion can be difficult to sort out. Early on, problems may be missed by the person with the concussion, family members, or doctors. People may look fine even though they are acting or feeling differently. More than 38 million boys and girls, ages 5-18, participate in organized youth sports across the country. And while sports can be a fun way to socialize and instill values such as teamwork, physical activity can also mean potential injuries. Concussions are one of the most commonly reported injuries in children and adolescents who participate in sports and recreation activities. The Centers for Disease Control and Prevention (CDC) estimates that as many as 3.8 million sports- and recreation-related concussions occur in the United States each year. The CDC also estimates that one out of five high school athletes suffer a concussion each season. TBI is a contributing factor to a third (30.5%) of all injury-related deaths in the United States. For that reason, I have developed a comprehensive monitoring and treatment program for anyone who may be at risk of experiencing TBI during their lifetime.

### SUMMARY OF THE INVENTION

The enclosed invention(s) include an accelerometer array as headgear that is fitted inside a “skull cap” or thin antimicrobial elastic cap or headband. This accelerometer array is intended to indicate when a concussion may have occurred in real or near-real time during practice or game for any high impact sport. Since accelerometer arrays are not very accurate at determining concussions, the same headgear or additional sideline tests/measurements involving ultrasound, EEG, biomarket, or other concussion detection test should be performed once the accelerometer array indicates there was an impact that may cause a concussion. Once a concussion is indicated or determined by further testing or analysis by a coach or athletic trainer, the athlete can then use an Internet based system that will capture sensor/test information and manage them and other stakeholders through the entire care and recovery process. This process may include online physician visits and analysis, issuance of prescription drugs for the injury, and guidance to have the athlete visit a hospital or TBI trained clinic for further care during the recovery period. Once the athlete has healed and is ready to return to a normal education and physical activity lifestyle, the same Internet enabled system will also record the release statement from the physician or athletic trainer providing care and will walk coaches, athletic trainers, parents and athletes though a program that will gradually increase both physical and mental activity until a normal lifestyle is achieved. The same system has additional sensors for detecting core body temperature, hydration levels, GPS for measuring acceleration, deceleration and position on the field of play for further analysis, and can also provide care and recovery services for overheating, dehydration, and other injuries associated with such conditions. In addition, similar sensor arrays to monitor blood glucose levels in a non-invasive manner as well as monitoring for maintenance of cardiovascular and neurological disorders and related injuries with or without online care in concert are detailed in this disclosure.

### BRIEF DESCRIPTION OF THE DRAWINGS

FIG. 1: Potential Hardware Design Layout for Head Trauma Measuring Devices

FIG. 2: Additional Hardware Design Layout for Head Trauma Measuring Devices

FIG. 3: Standalone Hardware Design Layout for Head Trauma Measuring Devices

FIG. 4: Possible System Flow Chart for the Process of Monitoring and Providing Care for Patients

FIG. 5: Possible System Flow Chart for Providing Internet Enabled and/or Physician or Athletic Trainer Care for Patients

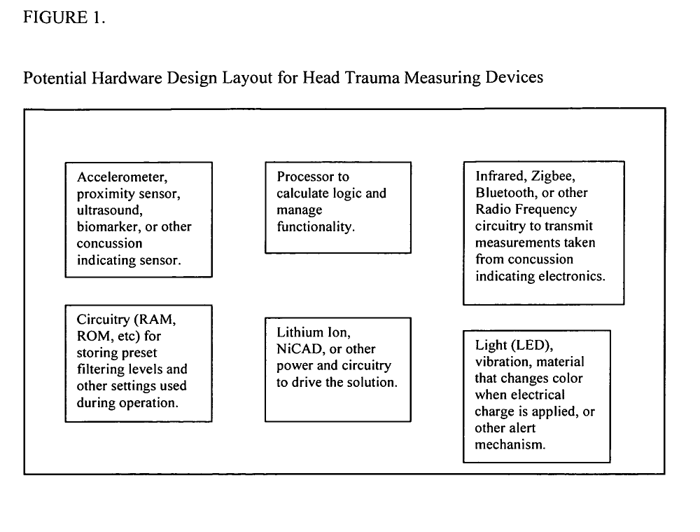

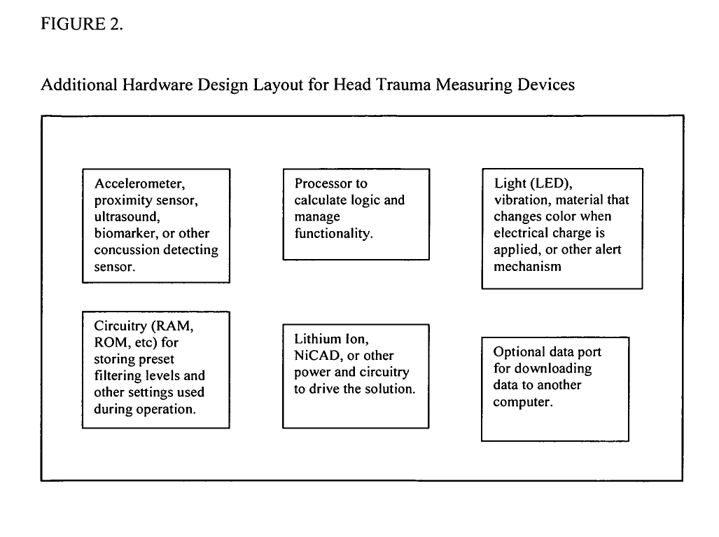

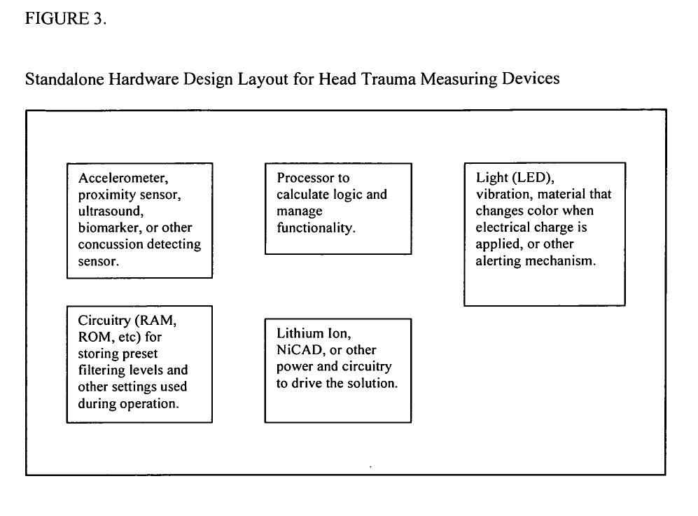

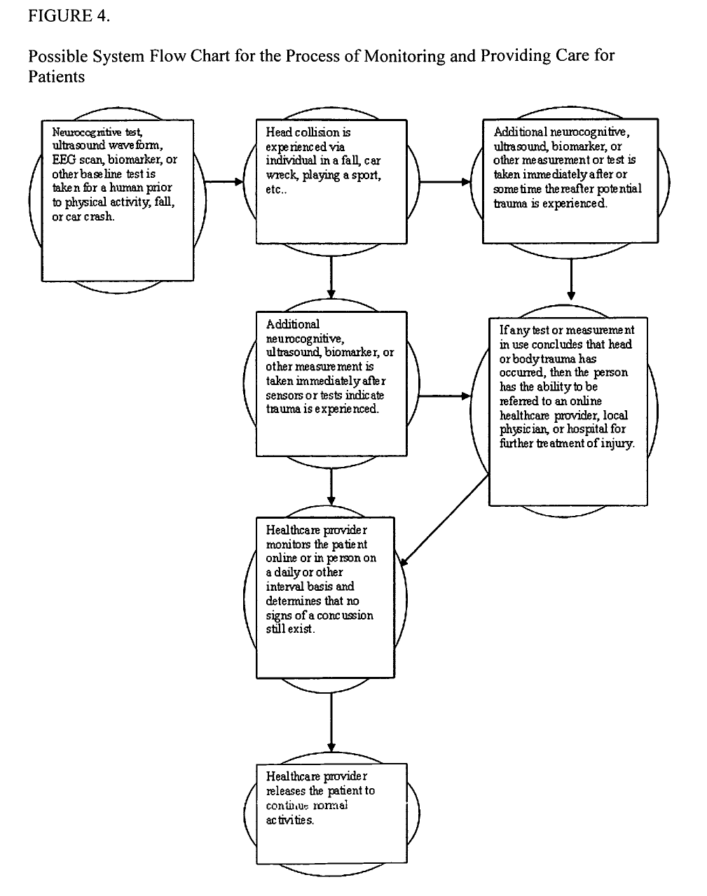

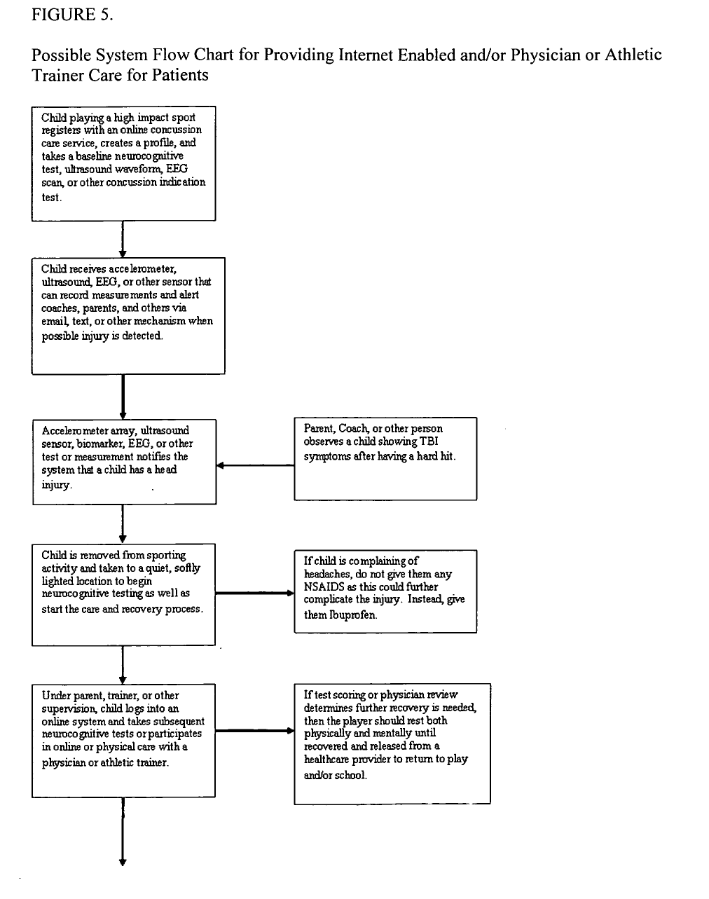

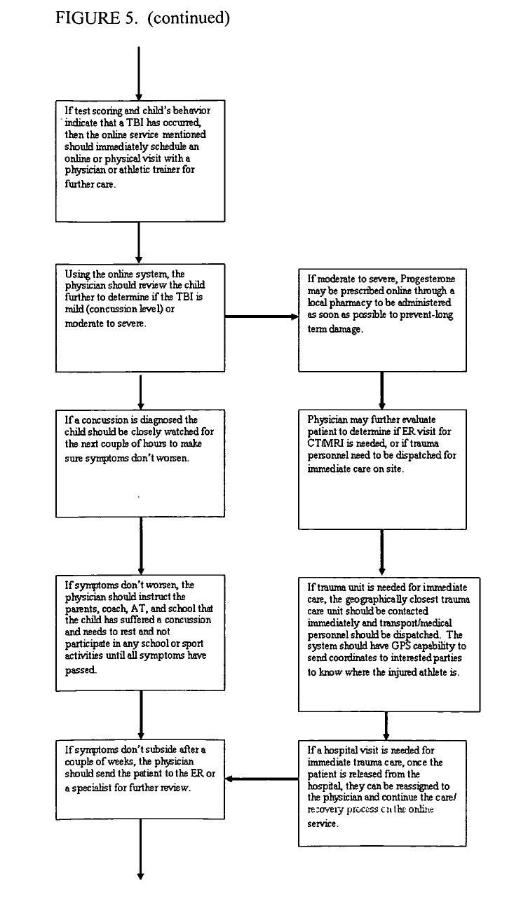

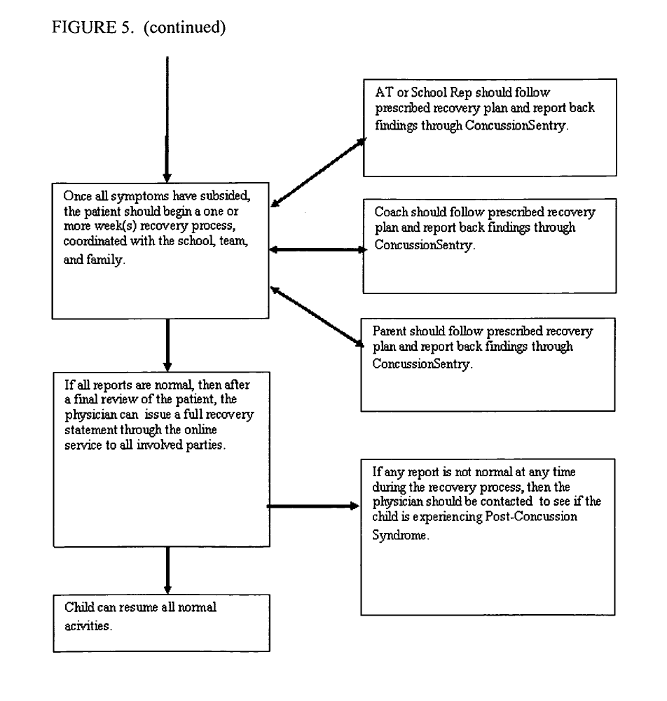

### DETAILED DESCRIPTION

The Internet enabled care and recovery program consists of a technology platform that utilizes groundbreaking new technology in identifying TBI and working with healthcare providers in the TBI field. In some cases, it utilizes wireless and remote monitoring equipment in concert to provide additional necessary care for individuals. In addition, the Internet enabled care and recovery TBI program offers all the necessary testing and healthcare provider monitoring to comply with the recommendations made in recent years by the best TBI experts in the field. The system is designed to care for TBI related injuries throughout the life of the patient, regardless of which sport they participate in or which league they are playing in.

For this discussion, the electronics equipment that a participant in the program may wear during physical activities will be referred to as the sensor array. The sensor array's primary purpose will be to determine if a wearer of the technology has potentially had a concussion or other vital sign change which warrants medical attention or special care. The sensor array will have several designs, all of which take the form of a headband that is designed to measure head impact or other vital signs during physical activity. The headband will have a small area fitted most likely on the back of the head of the person using the equipment to contain computer hardware and sensors to determine the condition of a person during a particular activity (playing sports or other active pursuit). The Sensor array hardware will consist of a processor for handling logic, potentially additional memory for storing information during play, a G-Force sensor to measure shock to the head during play, and a battery storage mechanism to power the electronics during use. The Sensor array may also incorporate a data port to download data via a computer cable or wire to another network at some point during or after play. The Sensor array may use mechanisms such as light (LEDs) or vibration to notify the user or other people that they have had a shock severe enough to potentially have caused a concussion. The headband may also incorporate paint, dye. or material that will change color when an electrical charge is applied to notify the user when they have had an impact that may be at a level that would cause a concussion. Each indicating mechanism may also change based on the level of impact. For instance, if the user has a severe impact, then the light may flash in a different manner than if the readings from the G-Force sensor indicated a mild concussion. This could also change the vibration pattern or color pattern accordingly to further provide information regarding a collision in realtime. The overall design is modeled so that any person participating in any physical activity can use the equipment. If the physical activity involves a helmet, the headband may be constructed in a manner that will allow it to be worn underneath the helmet or built into the helmet. If a person participates in activity that normally doesn't use a helmet, such as soccer, volleyball, etc., then the sensor array can be worn as a regular headband and can be made to look like such an item. The other benefit of Sensor array is that it can be used across all physical activities played and at any level of age to provide TBI care and also keep an overall record of all related injuries in a single data center for use in the future.

One Sensor array model, considered the active updating model, will have the capability of updating to a short or long range RF transceiver. The short range active updating model will require a wifi, zigbee, or other short range radio chipset built into the headband that will send out G-Force readings once they have passed a certain threshold to the radio transceiver (like a WiFi router or smartphone with WiFi capability) on the sideline or near the playing area and store or relay that information to a wide range network like a cellular network (GSM, CDMA, TDMA, or similar mobile device network) or satellite network. The information can also be relayed from the transceiver via a land based phone network. The information transmitted will be sent to a data storage facility for permanent storage for patient, parent, coach, trainer, or physician to review and use in the future throughout the person's life to provide care related to the concussion. The long range model will incorporate a modem that can send and receive data to be built into the head band itself and send the information through a wide area wireless network like a cellular network (GSM, CDMA, TDMA, or similar mobile device network) or satellite network to the data center.

Another sensor array model, considered the passive updating model, will not have the capability of updating to a short or long range RF transceiver. Instead, it will store G-Force information related to concussions on a memory chip on the headband itself so that it can be downloaded from the headband at a later date (most likely after a game or practice) to be evaluated and used for indicating a concussion may have occurred. This download can occur by incorporating a mini-USB or other serial data port on the headband and plugging the headband into another computer to have the computer receive the information and send it to the data storage facility mentioned above for immediate or future care of the individual using the ConcussionBand.

Yet another sensor array model, considered the basic model, will not have the capability of updating to a short or long range RF transceiver. It may or may not have a data port available so the information can be downloaded to a computer. Instead, its primary purpose will be to simply notify the user or persons with them that they have had an impact that may have caused a concussion. The mechanisms for notification include any of the three notifications mentioned above (light, vibration, or color change) to indicate that the user has potentially experienced a concussion related impact.

Each of these classifications of models may incorporate ballistics or impact gel, foam, or any other impact reducing material into the headband to protect the user during impact. They may be built out of a material such as cotton, any synthetic and/or antimicrobial material, or any other suitable material for wearing on the body during physical activity. The electronics should be designed in a manner that is as lightweight and discreet as possible to the user. One design may use a plastic shield between the electronics and the user in a manner that improves comfort. The design may also use a layer or strips of ballistics gel or other similar material to reduce impact with the skull or in a manner that may prevent skin breakage during an impact to the back of the head. The electronics should be designed in a manner that minimizes any heat generation during use from the electronics, the headband, or the person's head itself to improve comfort. The headbands can also have custom paint and/or dyes used to display the person's favorite sports team, team playing for, or any other image(s) that the user wants. They may be made of an all elastic material or out of a material that can be adjusted to better fit an individual. The individual may also want to have additional information displayed on the headband including their name or player number.

The data storage facility mentioned above should perform in the following capacity. One purpose is to send out email or text messages to interested parties when a person has experienced a concussion or other altered state. Another purpose is to store information related to the impact that occurred. This information may be where the impact occurred, what part of the body was hit, which team the player was playing with, what were their symptoms if any that occurred immediately following the collision, how long were those symptoms felt, the person's baseline ImPACT or similar concussion related test score, the person's test score(s) after the suspected concussion occurred, the treating healthcare professional's comments and record of when the person was cleared by the healthcare professional and why. Additional information such as the number of concussions, the G-Force measurements during impact, and the overall healing timeframe and symptoms can be extracted and used by the medical industry to data mine for potential future treatment and recommendations for care related to concussions.

One possible mechanism of the program involves post-concussion care aspects. This mechanism is described in the next section, which involves online and referral treatment options for an individual who is suspected of having received a concussion or other TBI.

The post-concussion care program is implemented first by having the individual take a baseline test to register normal overall cognitive and physical capacity before any physical activity begins. This test should be taken as soon as the person gets involved in the program. It measures ability in thinking, sensation, language, or emotions along the following factors:
Thinking (i.e., memory and reasoning);
Sensation (i.e., touch, taste, and smell);
Language (i.e., communication, expression. and understanding); and
Emotion (i.e., depression, anxiety, personality changes, aggression, acting out, and social inappropriateness).

This test should be done for people playing sports at all levels (youth, high school, college, and pro) who want to participate in the program. One baseline test offered is the ImPACT test, which is already used throughout the NFL and NHL. However, other tests can be provided for baseline testing depending on the situation and level of play. Once the baseline test is completed, this information is permanently recorded in an online database for future use and analysis.

Next, the player may be outfitted with a small, lightweight, and cost-effective force monitoring solution referred to above as the ConcussionBand. The solution is encased in a thin plastic shell and placed inside a Neoprene headband for sports where no head protective gear is used (soccer, volleyball, lacrosse, etc.) or can be fitted inside any helmet or head protecting equipment in use in a specific sport (football, hockey, etc). The hardware is powered by a NiCad or lithium-ion battery and can be rechargeable and measures G-Forces occurring during an impact. Once certain G-Forces are experienced (exceeding 80-90 Gs of shock is believed to be enough to cause a mild concussion), then the equipment sends an alert to the sidelines during a game or even practice to notify trainers and coaches that the player has had a significant impact. Once they receive the alert, they can then take appropriate action and remove the player from the field of play. If the Sensor array hardware is not used by players, they can still participate in the program once a concussion has been deemed to have occurred.

The next phase of the system is to have the player take a Internet enabled care and recovery online test in the locker room or at some other location (a school classroom or someone's home) to further determine whether or not the player has indeed had a concussion and to measure the severity of the damage being experienced. This testing can be compared against the baseline testing done previously to determine if the player's capacity has been diminished enough to determine a concussion or further TBI has occurred. If a TBI has occurred, the player then becomes a Internet enabled care and recovery TBI patient and is referred to appropriate healthcare providers for further examination.

The Internet enabled care and recovery system then provides a mechanism whereby all players can receive the same high quality healthcare that they would receive if they were to visit a Concussion Center, of which only a handful exist in the U.S. today. By purchasing the Internet enabled care and recovery program, the patient is then overnight shipped a webcam for use with a computer at home, school, or other appropriate location for further examination. The patient will then be assigned a healthcare professional specifically trained in caring for people who have recently experienced a TBI. Since the treatment for concussions is primarily to get both mental and physical rest, the patient should follow that advice but come back into the website for further evaluation during the recovery period. The healthcare provider will interview the patient on scheduled intervals to further test and observe the condition of the individual until they feel as though the patient has returned to full mental and physical capacity. Once this is determined, the healthcare provider can then clear the patient for further play but the patient is required to rest one additional week after their condition has returned to normal. If during this one additional week of rest, the patient has any relapse in concussion-related symptoms, they should contact their assigned healthcare provider immediately for further care. If the patient has had one full week of normal activity with no recurring symptoms, then they can return to play with a letter of good health from their healthcare professional.

The Internet enabled care and recovery system is a significant breakthrough in standardizing healthcare for TBI, while offering superior technology to assist in identification of and care for TBI related injuries. The player doesn't have to worry about whether or not they have to determine if they have had a concussion, and the coaches and parents can have “peace of mind” knowing that the most advanced techniques in identifying and caring for patients with TBI are being used. The additional benefits include significant cost savings over having to visit expensive hospital emergency rooms to get this level of care. Collectively, the Internet enabled care and recovery program is a solution that all participants of physical activity should participate in.

The monitoring of concussions can also occur in an unobtrusive manner during car accidents by incorporating proximity and tactile or pressure sensors to the cockpit of an automobile. In this scenario, passengers can be measured for force against the steering wheel, headrest, seat, or other part of the vehicle to determine force applied to the head or body. In this manner, the system can then determine if enough force was applied during impact to cause a concussion or other TBI. In addition, proximity sensors could be used to measure the speed at which the head or body of a passenger moved during the crash to also determine overall force applied to the person. These mechanisms can be used separately or together to determine if the person in a vehicle may have suffered a TBI. They can then also use the system to gain proper post-concussion care and keep all related injuries in their profile on the data center for later use.

Additional derivative models may include sensors that may be incorporated into alternative or models mentioned above to measure blood pressure, core body temperature, muscle fatigue, and/or overall hydration levels. These derivative models may not be headbands, but may use the same materials used in the overall headband construction for monitoring vital signs on other parts of the body such as the leg or arm. Each of these models may incorporate some or all of the techniques above to monitor vital signs and notify the user or others that the user of the technology has surpassed normal vital sign conditions and should be treated accordingly. They may be used to monitor and assist lifestyles of people with other neurological disorders including Parkinson's, Alzheimer's, Epilepsy, Lou Gehrig's Disease as well as Muscular Sclerosis, Down's Syndrome, etc.

In addition to putting electronics into a headband, they can also be placed inside shoes, padding worn by the user (shoulder pads, thigh pads, ankle pads, etc.), or as a wrap around any part of the body in addition to the head. Other places for the sensor monitors to be located are on the leg, ankle region, or arms/wrists. The mechanism can include one or more sensors in an array to monitor multiple body vital signs. Such sensors may monitor temperature, blood pressure, heartbeat, or muscle fatigue. One mechanism to monitor muscle fatigue could be using a tactile sensor or other surface pressure monitoring electronics to determine if a muscle is fatigued. One ideal place, for say a person with MS, could be the thigh to measure the largest muscle in the body for fatigue since it may fatigue faster than other muscles. The tactile sensor can be used to determine elasticity of the skin or to determine the tenseness/tightness of the muscle itself to indicate fatigue levels. This technique could also be used in sports to determine when an athlete is coming close to fatiguing a certain muscle group during practice or play. This would be useful if the player has sustained injury to a part of the body and needs to monitor that part of the body for overuse. Once certain fatigue levels are reached, the player can then be removed and allowed to rest.

The equipment may use a non-rechargeable battery source or a rechargeable source. If a rechargeable source is used, then the receptacle used to plug into the device to recharge should be able to be sealed so that moisture/dust/etc. can't get into the device and damage it. One other mechanism for recharging could also be a mechanism used on IPhones currently, where the device can be placed on a charging pad to recharge the internal battery without having to physically expose charging wiring to the outside of the device. This would allow the device to be recharged without having to worry about damaging a charging port during use.

One other possible usage of this solution could be the use of proximity sensors for use with someone who has bad vision or is blind. Proximity sensors could be placed on different parts of the body including the wrists and ankles to notify the user that they are approaching an object. This may give the user a 3 dimensional perspective on their surroundings. For instance, if the user is approaching an object such as a curb, then the ankle sensors can vibrate or make a sound such as a soft chirp to let the user know that they are approaching a low object. Another scenario is that if they are approaching a building or wall, all sensors can “go off” and indicate to the user that they are approaching an object that they can't go through, under, or over and need to go around. Sensors can chirp louder or more frequently or vibrate more when the user is getting closer to the object or an object such as a car is approaching as well to assist the user. This approach of sensors indicating when objects are getting closer or further, and where their position is in regards to the user, can eliminate the need of a walking cane and potentially allow a person with impaired or no sight to live a more normal and safer life.

The data center used in the initial discussion should take on all features of the Internet based mobile service platform provided by Archetype, Inc. of Birmingham, Ala. whereby users can be tracked historically and reports can be generated to provide information and metrics regarding the sensor based solutions and the subsequent treatment can be tracked permanently for use by a doctor, the medical industry, a coach, trainer, or insurance companies for various reasons. As an enhancement to a sensor mechanism that monitors blood pressure, heart rate, or body temperature, potentially core body temperature, concussions, and/or muscle fatigue, this information can be stored and retrieved for later use. The system can factor in other items at a later time period such as outdoor temperature at the time of physical exertion (practice or game) and predict when the user may fatigue, overheat, etc. Coaches could then use this information to decide on which players need to be kept under closer watch for overheating, concussion, and/or physical fatigue on the field, and this information could be used to determine how much practice/playing time the player gets as well as which position they need to play as a result of historical trends associated with that person's overall capabilities. The information is also recorded so that it can be used while the person is in youth sports, high school, college, and amateur or pro play as an adult and provide a seamless level of care for the individual throughout their lifetime. Having GPS integrated into the wireless router on the side of the field or incorporated with the data sent to a smartphone or similar device can also be used to notify emergency personnel of where the individual is in case the system detects a life threatening or serious injury where emergency care is deemed to be needed. All the information recorded by such a system can be court admissible so that it can be used in legal proceedings in the future if needed. The same treatment and care mechanisms mentioned in the earlier filing last week can also be used to care for all mentioned conditions and illnesses. This includes patient referral and recording of vital signs for use in care when future complications arise.

By integrating user notification such as vibration, change in color, or sounds into the mechanisms used/worn by the individual, then the devices can be used to notify the user that their vital signs aren't normal even when they are working alone or far enough away from a transceiver to be detected. Any other mechanism may be used to notify the user without the use of additional equipment besides the sensor layout or that can't be worn on the body or easily carried around by the user. This would include any notification including touch, sound, or visual notification, and may incorporate a sensor layout communicating with a smartphone worn by the user, or have the vibration, sound, or visual notification incorporated into the sensor housing itself.

Readings from Sensor array should allow a healthcare provider or the system itself determine if the person has a MTBI or a more severe head injury. If more severe head injury is suspected, the person should be referred immediately to a proper healthcare provider for further testing (CT, MRI, etc) to determine the severity of the injury. If the condition is deemed to be a mild TBI, or concussion, then the person can enter the Internet enabled care and recovery program and begin with the ImPACT or similar testing to verify the injury.

The person may be allowed to choose a particular sport and/or overall physical ability during normal play/activity once their symptoms have dissipated and they have been cleared by a healthcare provider to return to play. The person can then be provided a daily checklist of recommeneded activities to return them to full play/activity ability. This can be supervised by a parent, trusted person, coach, or athletic trainer for the sport/activity chosen to monitor their progress and record the achievements made to build up to full play capacity. The checklist can be completed prior to the player returning to full play to complete the treatment for that injury. A healthcare provider, coach, trainer, etc. may/may not be needed to review the checklist prior to return to the chosen game/activity. All the review for this part of the program can be done in person or online via the Vitalcare website. Every aspect of this system should be modified accordingly if needed to accomodate changes recommended by the medical community as improved treatment plans are developed. The waiting one week may be needed, but the healthcare provider should make the final recommendation as to whether that period for recovery once vital signs are normal is enough time passed to allow the player to return to their normal activities, work, or sport. Some people may take longer or shorter to be cleared for normal activity, work, or sport.

The G-Force measurements can be used to distinguish between mild, moderate, and severe impacts. If the impact warrants, the system should be able to provide immediate notification of emergency personnel available on the sideline or through contacting 911 emergency centers or other medical personnel. This should be done in a capacity as to have medical personnel respond as soon as possible. The system should be able to respond accordingly and notify appropriate medical personnel immediately if needed.

In addition to supporting accessing the information collected to determine force applied to the brain via the Internet, the information collected by the system referred to as VitalCare should also support downloading the data to devices such as local computers, laptops, smartphones, or other computing devices that can display and further enhance usage of such information. These tools could also support combining information such as weather reports with the collected information to provide additional reporting and forecasting capabilities with regards to the player's activities.

Tests used to verify concussions or other TBIs can be any form of neurocognitive test can be used to not only identify the concussion after a Sensor array indicates a concussion level force or greater has been received. These same or similar tests can be used to reevaluate the person when needed or on scheduled intervals either on the Internet or via a computer based software program on the sideline, lockerroom, office, etc. to determine if neurological patterns have returned to normal. These tests should be compared against a baseline reading or similar readings of neurocognitive functions taken when the person is normal and healthy.

In addition to administering reviews of patients, healthcare professionals can offer posted or online information regarding concussion management overall, or provide specific guidance to an individual via email, postings to their account, web conferences, phone calls, or other forms of communication held when needed. Parents, coaches, teachers (if they are in school) and other interested parties should be able to access or provided all information if needed or required. The recovery program should consider all activities, such as school, in putting a proper plan in place. The person recovering can then check off all activities performed during their recovery period and all associated side effects of the concussion on an ongoing basis.

The neurocognitive tests offered can and should be an improvement over current options that require up to 30 minutes of test time with review after the test is over. The primary improvement should be that the test should be as brief and efficient on time as possible to get the review done in the shortest time possible. In doing so, sideline testing could allow the player to return to play if cleared by the healthcare provider in charge for the same game the concussion occurred in. In addition, the testing could be interactive with the player and healthcare provider so that additional review after the testing is over is eliminated as it would be done during the testing to expedite the player returning to play.

In addition to the neurocognitive testing, the online/offline resources provided can provide additional training and testing for physicians, healthcare providers, coaches, managers, parents, and the person using the system themselves to educate themselves about concussions. The services can also provide audit trails of educational plans and provide reports to appropriate groups including insurance companies, federal/state/local government agencies, schools, parents, sports associations, and coaches as to who has been properly trained and educated to deal with this issue.

One significant breakthrough will be to be able to perform a blood test on sit where the injury occurs or at a facility soon after that will identify whether or not a concussion occurred, as well as the severity of the injury. Once that is possible, the system can be used to prescribe Progesterone or other drugs online within minutes of the injury to reduce the short and long term damage associated with these injuries.

The system should also allow potential new concussed patients to use the care portion even if the athlete hasn't performed a baseline or recent neurocognitive test. To improve the quality of the results, the patient should be able to load a percentile ranking score into their profile from any standardized, IQ, or other test taken in the past that can represent cognitive capacity. The standardized score should be normalized into a score that is deemed equivalent to the neurocognitive test offered by this service. Then, the neurocognitive score taken from the postconcussion test can be compared to the standardized, IQ, or other test score taken before the concussion to determine if neural capacity has been reduced indicating a TBI. This will dramatically improve the accuracy of a test score taken after a concussion where no baseline test is available. The system should also allow the physician to issue prescriptions for depression, sleeplessness, or other side effects of the TBI as needed during the care and recovery process to patients.

The biometric/online care system that uses biometric sensors to detect sports injuries and neurocognitive illness complications and provide physician guided Internet available care can also monitor and care for cardiovascular conditions. The cardiovascular sensor system will monitor heart disease, bypasses, and other cardio related conditions/illnesses for blood flow tracking, mini-strokes, strokes, and other items that may lead to complications. This includes cardiovascular illnesses, diseases, surgically implanted devices and their performance (including stints, bypasses, pacemakers, etc.) for proper operation. If a heart attack occurs, the same system may include a mechanism that can deliver a dose of aspirin automatically into the bloodstream or digestive system of the person that has had the attack. This is done in order to help the person survive the heart attack, and has proven to allow 23% of the people who take the recommended dosage right after a heart attack is suspected to survive the heart attack. The FDA has approved taking one-half (160-162.5 mg) of a regular-strength aspirin tablet for dosage administered after a heart attack has been suspected. In addition, this same mechanism could also be used to help stroke victims with just aspirin or additional medications to assist in recovery. This medicine delivery system more than likely will utilize some kind of needle mechanism that can allow medicines to enter the blood stream through the skin, but it may also support other forms of delivery through the skin or digestive system. For people who have suffered a severe head injury, progesterone may be administered through this system. Other medications that should be administered to correct a bodily function if something goes awry, such as a diabetic or person with epilepsy having a seizure, can also be administered if a seizure is detected by the biosensors (most likely an accelerometer to detect shaking).

This system can be used in a manner where insurance claims are submitted each time care is provided online. The care may be overseen by a physician or other trained personnel, or may be an automated process already approved by a physician or association for usage.

The biometric sensors can work as standalone hardware apparatus and buzz, vibrate, or speak to the user to let them know there are problems occurring. The system can also change colors or perform other notifications on the person to notify them or someone else that they are having health problems. The sensors can also be combined with an RF implementation attached to the person using them. This could be a mobile phone with Bluetooth communicating with the sensors placed elsewhere on the body such as the head and chest, or it could be a self-contained hardware implementation where the sensors and the RF communication are all part of the same physical part. The communications used could be Zigbee, WiFi, GSM or any other short or long range radio device. If short range (Zigbee, WiFi, or Bluetooth) is used, then there could be a receiver close by on, for instance, a football or soccer field that could relay the data to a centralized data storage facility. In addition, the short range device could send data to a wireless modem that would receive the short range signals and then retransmit them through a long range radio network such as a mobile phone network. The third implementation could combine the sensors with a long range radio communication chipset such as GSM to operate across the same area as a mobile phone would as a single self-contained unit. All three sensor implementations (standalone, short range wireless with long range relay nearby, and long range wireless transmission) should be accommodated by a single vendor to accommodate a large group of possible users, such as all NCAA or NFL players.

The medicine delivery mechanism could also be used to deliver ongoing medicine doses to people who have to take the same medicines in a controlled interval (such as daily or weekly) for maintenance of their illness or disease. The medicines could be put in a container in a concentrated form to save space and make the container less evasive than full size doses would be. Since the system will have computer timer capability, a means by which to administer controlled doses of medication to people who otherwise can't or don't have the mental capacity to take them may be utilized. The drugs will be secured in concentrated doses and can be loaded as cartridges once a month by a caregiver. The doses could potentially be modified by the physician over the Internet as needed for proper care. The central data storage system needs to track all current medications being administered and allow the physician to modify any combination of these doses over the Internet to the patient hardware directly to control and change the medications prescribed as needed. An EEG helmet can also be used to track where and when Epileptic seizures are occurring and to what frequency and degree of intensity so that the physician can have a more immediate and ongoing analysis of the condition of the brain. The same mechanism can be used to monitor deterioration or improvement of brain activity of illnesses/injuries on a daily basis for the patient. The Diabetes implementation, as well as all the neurological implementations, should be able to detect seizures and/or heart attacks and/or strokes for each patient. This same implementation should be able to detect blood glucose levels without having to penetrate the skin, as well as perform other blood monitoring processes related to each illness that will assist in care of the disease or illness. These biometric monitoring systems can be used to build up a database of proper care mechanisms of people based on age, gender, height, weight, and other traits across groups being provided care. Once the database is built up, it can be used along with any of the already defined biometric sensor layouts to assist in diagnosis of an illness where symptoms such as seizures, increased blood pressure, or other vital sign measurements can be used to diagnose a patient for a yet undiagnosed illness, disease, or injury.

One significant breakthrough will be to be able to perform a blood test on site where the injury occurs or at a facility soon after that will identify whether or not a concussion occurred, as well as the severity of the injury. Once that is possible, the system can be used to prescribe Progesterone or other drugs online within minutes of the injury to reduce the short and long term damage associated with these injuries.

The system should also allow potential new concussed patients to use the care portion even if the athlete hasn't performed a baseline or recent neurocognitive test. To improve the quality of the results, the patient should be able to load a percentile ranking score into their profile from any standardized, IQ, or other test taken in the past that can represent cognitive capacity. The standardized score should be normalized into a score that is deemed equivalent to the neurocognitive test offered by this service. Then, the neurocognitive score taken from the postconcussion test can be compared to the standardized, IQ, or other test score taken before the concussion to determine if neural capacity has been reduced indicating a TBI. This will dramatically improve the accuracy of a test score taken after a concussion where no baseline test is available. The system should also allow the physician to issue prescriptions for depression, sleeplessness, or other side effects of the TBI as needed during the care and recovery process to patients.

The system also can incorporate an additional sensor design that uses an accelerometer array alongside with other technologies to more accurately indicate concussions in real or near real time. The idea here is that we use the somewhat inaccurate accelerometer array to be a low power trigger that will initiate an ultrasound, EEG, biomarker, or other test/measurement. That way, we can have equipment that can be worn by the athlete during practice and/or a game with a small power source for comfort and we can also incorporate the more power intensive but more accurate ultrasound, EEG, biomarker, or other test/measurement to provide a filtering mechanism to reduce and hopefully eliminate many if not all false positives and/or negatives that occur while using accelerometer based indication solutions currently. The additional filtering test to make the alerting or reporting more accurate could occur as an additional step and not require the sensors/test equipment used to perform the more accurate assessment of the injury to be worn on the athlete during play. This means the ultrasound, EEG, biomarker, or other test/measurement to determine if a concussion or other TBI occurred could be maintained on the sideline, locker room, or on a hospital or clinic for use after an impact is indicated by the accelerometer array to more accurately determine the extent of the damage.

There is now research that indicates that the rotational acceleration of the head during impact and the combination of rotational and linear acceleration are far more important factors in determining the concussion than linear acceleration alone. The latest research indicates that we need to be looking at linear acceleration, rotational acceleration, duration of impact or HIS, and another measurement which is simply factoring in historical impacts and their outcome per athlete.

During practice or games as collisions occur, the concussion or other damage to the brain from an impact almost always occurs when the force of the impact allows the brain to shift enough to impact the inside of the skull. The impact itself or shearing of tissue is what causes the injury. If that is the case, then one would think there has to be some unique sound resonations that occur when both events take place. Considering that pretty much all human brain tissue is of a certain density, fluids around the brain are of an identical chemical structure, and skulls are all made of course of bone, shouldn't there be some highly unique sound resonations or vibrations that occur when tissue shears or the brain impacts the inside of the skull. In other words, if we put directional short range audio receivers or vibration sensors around the skull pointed toward the brain of the athlete that would listen for specific sound wavelengths, specific ranges of sound wavelengths, or vibrations that correspond to those events, theoretically we should have a highly accurate indication that damage has occurred. Even better, we can simulate such sounds and/or vibrations in a lab with modeled prototypes of human heads or use fresh cadavers to determine sounds generated from the brain impacting the skull. That way, we don't have to wait and have a lot more concussions occur for further research to mature some other way of accurately identifying a concussion. We may be able to “tune this”, pardon the pun, to filter out mild impacts that may not cause any injury versus significant brain/skull impacts that would more than likely cause damage. This could lead fairly quickly to what seems to be an exceptionally accurate determination over existing concussion detection systems because we can easily recreate the sounds generated when an impact occurs in a lab. With such a detection system, we should also be able to listen for or detect any shearing of tissue to detect subtle injuries that don't necessarily involve the brain impacting the skull. We could also easily reproduce the sounds or vibrations produced from the shearing for detection of injuries from shearing alone.

The care and recovery process may involve other people on the healthcare provider side as needed to assist in that portion of the process.

The care and recovery team may include:

A Physiatrist (Medical Doctor) who specialize in physical medicine and rehabilitation. The physiatrist coordinates treatment to maximize the level of function and is responsible for medical evaluations and plans of care most suitable for the individual and his/her family.

The Physical Therapist works with both the individual with TBI and their family to assist the injured person in becoming as physically independent as possible. If physical therapy is recommended, the PT's initial visit will include evaluation of the injured person's physical abilities. Physical therapy's goals not only include learning to walk, but also increasing strength, decreasing joint stiffness, improving balance and increasing mobility skills.

The physical therapist also evaluates patient's needs for equipment, such as, a wheelchair, walker, bedside commode and/or other items needed for use in the home.

The Occupational Therapist—works with the brain injured person to develop the skills needed to be independent with everyday activities. If occupational therapy (OT) is recommended, the occupational therapist will evaluate the patient to assess his/her skills which include visual, cognitive and perceptual abilities to perform tasks such as dressing, eating, grooming, bathing and homemaking (activities of daily living—ADL's).

The brain injured person's attention and concentration skills may also require training. Their ability to remember what to do first and how to solve problems will be evaluated. The ability to perform everyday tasks may require improvement in ability to use the hands and upper body. The OT plans exercises to help improve these areas.

Speech and Language Pathologist—The speech and language pathologist will evaluate the brain injured person's ability to communicate, including the ability to speak, understand, write and use hand signals. Often a person with a brain injury seems to understand much more than they actually do. It is important for the family and therapist to know how well the person understands spoken words so that instructions can be given in the best way. It is also important to know the best way to help the brain injured person communicate to reduce their frustration and stress. The person with a brain injury may also have difficulty with other thinking skills, such as sequencing, problem solving and judgment. Many times the brain injured person has difficulty with swallowing. The speech pathologist assesses their ability to chew and swallow foods of different amounts and textures.

Neuropsychology—The Neuropsychologist keeps track of the injured person's cognitive abilities (thinking skills and emotional status). Often after a brain injury, people have difficulty with basic thinking skills, basic memory, as well as reasoning skills. The Neuropsychologist evaluates the severity of disorder in these areas and can provide treatment as indicated. The Neuropsychologist also provides counseling to family members who wish to know more about brain injury but who may be having difficulty coping with family stress.

The Social Worker helps the injured person and family respond to social, emotional or financial problems resulting from the injury. The Social Worker can assist the brain-injured patient and his/her family gather information about local agencies that may assist the family and injured person with special services.

What is claimed is:

1. A non-invasive electronic sensor array comprising one or more accelerometers and an additional EEG test to indicate and assist in detection of a traumatic brain injury.

2. A non-invasive biometric sensor array comprising accelerometers

3. The array of claim 2, further comprising an additional audio equipment based test to indicate and assist in detection of a traumatic brain injury.

4. The array of claim 2, further comprising an additional biomarker test to indicate and assist in detection of a traumatic brain injury.

5. The array of claim 2, further comprising an additional test or measurement to improve accuracy of indication and detection of a traumatic brain injury.

6. The array of claim 2, further comprising heat sensors, and hydration sensors to indicate and assist in detection of any related sports injuries.

7. A method of using vital sign monitors, comprising a person or animal wearing said monitors in conjunction with Internet enabled physician or other healthcare provider services to facilitate care and/or recovery services

8. The method of claim 7 for an injury.

9. The method of claim 7 for a diagnosed medical condition.

10. The method of claim 7 for diagnosed cardiovascular medical conditions.

11. The method of claim 7 for diagnosed neurological medical conditions.

12. The method of claim 7 for a diagnosed medical condition.

13. A method of combining multiple biometric sensors, comprising combinations of measuring for G-Force, heat, and hydration levels on a subject, including or excluding online interfacing and care, unsupervised or physician based

14. A network architecture comprising utilizing sensors worn by a person or animal that are sent to local wireless transceivers (smartphones or industrial modems with or without integrated GPS) which retransmits data through long range wireless networks including cellular based (GSM, CDMA, TDMA, etc.) or satellite based (iridium, Orbcomm, etc.) to centralized data storage facilities.

15. A method of providing sensor based Internet-available reports and alerts for usage on-demand and in real-time.

16. The sensor of claim 1, further comprising alerting mechanisms when conditions are indicated may involve either email or text based alerting of individuals that may need to be aware of the sensor information.

17. The sensor of claim 2, further comprising alerting mechanisms when conditions are indicated may involve either email or text based alerting of individuals that may need to be aware of the sensor information.

18. An Internet enabled service, comprising sensors, neurocognitive tests, and/or physician care as a complete care and recovery process.

19. Headgear designs, comprising accelerometers, EEG sensors, audio sensors, biomarker sensors, and other sensors to be placed in a head wrap currently referred to as a “skull cap”.

20. The designs of claim 19, further comprising a gel or other shock absorption material that can reduce the impact force against the head during play.

21. The designs of claim 19, further comprising the method of changing the color of the headgear to indicate the player may have an injury.

22. The array of claim 1, further comprising Zigbee, Bluetooth, WiFi, or some other short range communication protocol to transmit alerts to a sideline receiver that can send the data through a wireless or wireline communications network for centralized data collection.

23. The array of claim 2, further comprising Zigbee, Bluetooth, WiFi, or some other short range communication protocol to transmit alerts to a sideline receiver that can send the data through a wireless or wireline communications network for centralized data collection.

24. Sensor implementations, comprising alerts to physicians or athletic trainers that drugs may need to be prescribed to a patient for a particular injury or medical conditions.
